(19) United States
(12) Patent Application Publication
(10) Pub. No.: US 2011/0099889 A1
(43) Pub. Date: May 5, 2011

(54) METHOD FOR PURIFYING BIODIESEL OR BIODIESEL PRECURSORS

(75) Inventors: Ulrich Sohling, Freising (DE); Friedrich Ruf, Ast (DE); Arno Cordes, Salzgitter (DE)

(73) Assignee: SUD-CHEMIE AG, Munich (DE)

(21) Appl. No.: 12/919,596

(22) PCT Filed: Mar. 2, 2009
(86) PCT No.: PCT/EP09/01468

§ 371 Date: Jan. 4, 2011

(30) Foreign Application Priority Data
Feb. 28, 2008 (EP) 08 003 709.6

(51) Int. Cl. C10L 1/02; C12P 7/00

(52) U.S. Cl. 44/307; 435/132

(57) ABSTRACT

A method for purifying biodiesel, biodiesel precursors, or the mixtures thereof, which contain at least one glycoside wherein biodiesel or a biodiesel precursor or a mixture thereof is incubated with at least one enzyme, in order to convert or cleave said at least one glycoside. In addition, the purified products which can be obtained according to this method and the use of at least one enzyme, which can cleave or convert glycoside, for purifying biodiesel or biodiesel precursors and the mixtures thereof.

### CROSS REFERENCE TO RELATED APPLICATIONS

This is a National Phase application of PCT application number PCT/EP2009/001468, filed Mar. 2, 2009, which claims priority benefit of European application number EP 08 003 709.6, filed Feb. 28, 2008, the content of such applications being incorporated herein by reference.

### FIELD OF THE INVENTION

The invention relates to a method for purifying biodiesel, biodiesel precursors, vegetable or animal fats and their mixtures, as well as the purified products available according to this method. The present invention also describes the use of at least one enzyme, which can cleave or transform glycoside, for purifying biodiesel, biodiesel precursors, vegetable or animal fats, as well as their mixtures.

### BACKGROUND OF THE INVENTION

Because of the limited occurrence of fossil raw materials and repeated increases in energy prices, fuels based on renewable raw materials are attracting ever greater interest. In particular, biodiesel is currently already being added to the diesel fuels available on the market in Europe. Additionally, vegetable or animal fats can also be used as fuels or serve as starting material for the production of biodiesel.

Biodiesel is produced by alcoholysis of triglycerides, wherein one mol triglyceride is reacted with three mols alcohol to one mol glycerol and three mols of the corresponding fatty acid ester. The reaction comprises three reversible reactions, wherein the triglyceride is transformed stepwise into a diglyceride, a monoglyceride and finally into glycerol. In each of the steps one mol alcohol is used and one mol of fatty acid ester released in each case. Methanol is used as alcohol in most industrial processes. However, biodiesel which contains ethyl or propyl fatty acid esters is also offered for sale.

The transesterification can be carried out as a one-stage process. It is, however, also possible to carry out the transesterification in several stages. In each step, only some of the required methanol is added and the glycerol phase separated off after each step. Additionally, the alcoholysis can be carried out under both acid and basic catalysis.

In most industrial processes the alcoholysis of the triglycerides is carried out under homogeneous alkaline catalysis. The alkoxide ion acting as catalyst is produced for example by dissolving an alkali alcoholate in the alcohol or reacting the pure alkali metal with the alcohol. In methanolysis, a corresponding alkali hydroxide can also be dissolved in the methanol. Because a phase separation due to the resulting glycerol occurs relatively rapidly during the alcoholysis of triglycerides, the great majority of the alkaline catalyst is removed relatively quickly from the reaction mixture. The resulting fatty acid esters therefore scarcely come into contact with the catalyst, with the result that the risk of saponification is small. Relative to the oil used, the catalyst is used mostly in a quantity of 0.5 to 1 wt.-%. For details of biodiesel production, reference is made to the monograph by M. Mittelbach, C. Remschmidt, “Biodiesel; The comprehensive Handbook”, Graz, 2004; ISBN 3-200-00249-2.

The triglycerides used as starting materials for biodiesel production can be obtained for example from vegetable or animal fat. Of the vegetable raw materials, four starting materials are principally used in the worldwide production of biodiesel, wherein rapeseed oil is the most important source, followed by sunflower oil, soya bean oil and palm oil. Further starting materials which are commercially significant are animal fats, such as beef tallow, as well as used frying fats.

In order to remove soaps produced during biodiesel production, as well as residual methanol, glycerol, mono- and diglycerides, from the biodiesel, in most cases a water wash is carried out after the transesterification. If the crude biodiesel contains large quantities of soaps a stable emulsion can form, which makes the separation of the fatty acid esters much more difficult.

Constantly increasing demands in respect of the product properties of fuels based on renewable raw materials are being made, both by consumers and by the authorities. In order to ensure a defined combustion of the biodiesel, in Germany for example limit values have been set for minor components in biodiesel. According to DIN standard DIN EN 14214, a maximum monoglycerides content of 0.8 wt.-%, a maximum free glycerine content of 0.2 wt.-%, a maximum diglycerides content of 0.2 wt.-%, and similarly a maximum triglycerides content of 0.2 wt.-% have been set.

However, although the methods of the state of the art already make possible a production of biodiesel or other fuels based on renewable raw materials which satisfy current requirements, the aim of providing consumers with improved fuels based on renewable raw materials is further pursued by a continuing programme of work. In particular the fuels based on renewable raw materials should contain no, or only minimal quantities of, disruptive constituents.

A known method of the state of the art for separating ingredients, such as for example sterylglycosides which can lead to disruptive clouding and precipitates in the biodiesel, is based on cooling the whole of the biodiesel to low temperatures and subjecting it to a filtration. This method is, however, extremely expensive to carry out. WO2007/076163 A describes methods for treating biodiesel with adsorbents and the like to remove steryl glycosides.

### DESCRIPTION OF THE INVENTION

An object of the present invention is therefore to provide a further method for purifying biodiesel, biodiesel precursors, vegetable or animal fats, as well as their mixtures, which helps to achieve a high-quality product. In particular such a method should help to separate off compounds which can lead to a precipitation of solids, with the result that a possible formation of precipitates can be avoided. Additionally, such a method should essentially be economical, and elaborate method steps, such as for example filtration at low temperature, avoided.

This object is achieved according to a feature according to aspects of the invention by a method for purifying biodiesel or biodiesel precursors as well as their mixtures, containing at least one glycoside, wherein biodiesel or biodiesel precursors or a mixture thereof is incubated with at least one enzyme, in order to transform or cleave the at least one glycoside. The present invention also teaches the use of at least one enzyme which can cleave or'transform glycoside for purifying biodiesel or biodiesel precursors as well as their mixtures. Furthermore the present invention provides biodiesel or biodiesel precursors as well as their mixtures which can be obtained according to the method according to aspects of the invention.

Preferred embodiments are given in the respective dependent claims.

Starting from the observation that in particular glycosides and, of these, above all the so-called sterylglycosides, for example sitosteryl-β-glucoside, but also e.g. cholesteryl glycosides can lead to clouding and precipitates, a reliable method has been developed by the inventors in numerous and elaborate tests which provides an extensive separation or transformation of glycosides in biodiesel or biodiesel precursors as well as their mixtures. This method does not require costly filtration after cooling, and can advantageously be carried out in some embodiments with merely one method step.

The term “biodiesel” and its meaning, as well as the fact that, during biodiesel production, initially lower-purity crude biodiesel can be obtained, is known to a person skilled in the art. Within the framework of the present invention the term “biodiesel” can also mean in particular any mixture of fatty acid alkyl esters. The alkyl residue of the fatty acid alkyl ester can for example be straight-chained or branched and comprise 1 to 28 carbon atoms. In particular the fatty acid alkyl ester can for example be a methyl, ethyl, propyl, butyl, pentyl, hexyl ester of a fatty acid. Preferably the mixture of fatty acid alkyl esters contains at least 70 wt.-% fatty-acid alkyl ester, preferably at least 85 wt.-%, preferably at least 95 wt.-%, in particular at least 98 wt.-%, in each case relative to the total weight of the organic constituents of the mixture.

Mixtures described as biodiesel can contain any quantities of mono-, di-, and/or triglycerides. Preferably, biodiesel can have a limited mono-, di-, and/or triglycerides content. For example the biodiesel can contain at most 2 wt.-%, preferably at most 0.8 wt.-% monoglyceridesi at most 2 wt.-%, preferably at most 0.2 wt.-% diglycerides, and/or at most 2 wt.-%, preferably at most 0.2 wt.-% triglycerides, determined according to DIN standard DIN EN 14214.

The purifying methods of the present invention can be used not only with biodiesel, but also with mixtures which are considered precursors for producing biodiesel or can occur as such. The term “biodiesel precursor”, as used within the framework of the present invention, denotes any mixtures which comprise monoglyderides and/or diglycerides and/or triglycerides of fatty acids. For example, such mixtures may contain at least 30 wt.-% mono-, di- or triglycerides, preferably at least 60 wt.-%, in particular at least 90 wt.-%, in each case relative to the total weight of the organic constituents of the mixture. Mixtures described as “biodiesel precursors” can also optionally comprise fatty acid alkyl esters or fats. The term “fat” can, within the framework of the present invention, mean any mixture which comprises triacylglycerides. By fat is meant mixtures with a solid consistency, semisolid consistency or liquid consistency at room temperature. In common parlance, fats which are liquid at room temperature are also called oils. It may be expressly pointed out that the term “fats” within the framework of the present invention comprises any oils such as for example the fats which, according to general current language usage, are also called soya oils, rapeseed oils, etc. below. A fat or a mixture of fats can be selected according to the general knowledge of a person skilled in the art. Fats of different origin and composition are for example listed in the “Lehrbuch der Lebensmittelchemie”, Berlin, 2001, 5^{th} edition, ISBN 3-540-41096-1, by Belitz, Grosch, Schieberle. It may be expressly mentioned, however, that fats which are contaminated or which occur as waste products, for example frying oils, can also come into consideration. According to a preferred embodiment the fat is a fat or oil with a lecithin content of less than 10 wt.-%, in particular less than 5 wt.-%, further preferably less than 10 ppm, in particular less than 5 ppm. According to an embodiment degummed and/or deodorized fats or oils are also preferred as well as biodiesel or biodiesel precursors with the above lecithin contents (phosphatidylcholine content). According to a further embodiment according to aspects of the invention fats or oils, in particular degummed and/or deodorized fats or oils, as well as biodiesel or biodiesel precursors with a glycero-phospholipid content, preferably a total phospholipid content, of less than 10 wt.-%, in particular less than 5 wt.-%, in particular less than 10 ppm, in particular less than 5 ppm, are also preferred.

Here, the term “glycoside” generally denotes compounds which consist of carbohydrates (mono- or oligosaccharides) and aglycones (i.e. non-sugars). According to a preferred feature the term includes compounds which result from a reaction of cyclic hemiacetal forms of aldo- or ketohexoses with alcohols accompanied by the formation of an acetal (“Lehrbuch der Organischen Chemie”, Stuttgart, 1988, 21^{st} ed., pp. 442 et seq., ISBN 3-7776-0438-0 by H. Beyer, W. Walter). Depending on the sugar forming the basis in each case, glycosides, as is known to a person skilled in the art, are for example called glucosides, mannosides, fructosides and, depending on the presence of a heterocyclic 5- or 6-ring, furanosides or pyranosides. The term “glycosides” also includes the oligo- or polysaccharides in which the glycosidic OH group is acetalized with a glycosidic or alcoholic group of an additional monosaccharide.

In particular the present invention makes possible a separation of sterylglycosides. Sterylglycosides are glycosides which, as is known to a person skilled in the art, are based on sterines. Sterines (frequently also called sterols) as such are nitrogen-free, polycyclic, hydroaromatic compounds, in particular derivatives of gonane or of perhydro-1H-cyclopenta[alpha)phenanthrene. An overview of sterols, from which the corresponding sterylglycosides can be derived according to the knowledge of a person skilled in the art, is found for example in: “Lexikon der Lebensmittel and der Lebensmittelchemie”, Stuttgart, 2005, ISBN 3-8047-2275-X by W. Ternes et al., pp. 1790 et seq.). U.S. Pat. No. 7,091,012 describes for example processes for producing sterols and polar lipids from vegetable oil lecithin fractions. As examples of sterylglycosides there can be named i.a. sitosteryl, stigmasterol or campesterol betaglucosides.

It is assumed, without the invention being bound by the correctness of this assumption, that the sterylglycosides are present in the educt material for producing biodiesel (for example animal or vegetable fats) in an acylated form in the 6-position of the glucose (i.e. in one in the original OH function at the C₆-position of glucose, in particular glucopyranose) and that, upon transesterification for producing biodiesel, a cleavage of the ester bond takes place, wherein a sterylglycoside with free OH group is formed in the 6-position of the glucose. (The numbering of the carbon atoms in sugar atoms is known to a person skilled in the art and is for example given in the “Lehrbuch der Organischen Chemie” (supra) by H. Beyer, W. Walter). The resulting sterylglycoside with free OH group has a poorer solubility in unpolar media than the sterylglycoside present beforehand in the starting material. The sterylglycoside transformed during biodiesel production can precipitate out at the end of the biodiesel process upon cooling to room temperature.

The present invention now provides methods in which, under enzymatic action, glycosides, in particular sterylglycosides, are converted by enzyme incubation into a transformation product of the glycoside and/or into at least one cleavage product of the glycoside. Unlike the methods of the state of the art which involve a precipitation of possible deposits or clouding through a drop in temperature, the present invention applies a chemical or biological transformation of compounds potentially leading to precipitations. This makes possible a specific reduction of undesired glycosides. All compounds in which the glycosidic bond between the sugar portion and the non-sugar portion of the glycoside remains intact can be considered transformation products, while all compounds in which the glycosidic bond between the sugar portion and the non-sugar portion does not remain intact and/or a section comprising at least one, preferably at least two or three carbon atom(s), is cleaved from the glycoside can be considered cleavage products.

A suitable enzyme can be chosen based on the knowledge of a person skilled in the art by taking into account the starting materials or mixture of starting materials available in each case. In particular the purity of the starting materials used and their origin (freshly obtained fats and oils or waste fats and oils) may need to be taken into account. Generally, all enzymes which display an activity for transforming or cleaving glycosides come into consideration. The activity for transforming or cleaving a glycoside, for example a glucoside, can be a main, but also a secondary, activity of the enzyme (such as for example with cellulases).

In particular enzymes which display an activity for cleaving an acetal bond and/or for cleaving a glycosidic bond between the sugar portion and the non-sugar portion can be used. There can be used as enzyme for example hydrolases, in particular glycosidases (EC 3.2.1 according to recommendations of the Nomenclature Committee of the International Union of Biochemistry and Molecular Biology (NC-IUBMB)), such as for example alpha-amylase, beta-amylase, exo-1,4-alpha-D-glucosidase (glucoamylase), cellulase, polygalacturonase, lysozyme, alpha-D-glucosidase (maltase), beta-D-glucosidase, cellobiase, beta-glucanase, chitinase, anthocyanase, naringinase, alpha-D-galactosidase, beta-D-galactosidase (lactase), beta-D-fructofuranosidase (invertase), 1,3-beta-D-xylanase, alpha-rhamnosidase, pullulanase, exopolygalacturonase, or their mixtures, with one or more further enzymes.

Particularly advantageous results were achieved for example when using glucosidases (EC 3.2.1), in particular beta-glucosidases, naringinases, β-glucanases, and cellulases.

In the case of a purification of biodiesel, preferably an enzyme which essentially does not attack ester groups can be used.

With the present invention, enzymes which display the activity of attaching an aliphatic residue, in particular a long-chained residue, to the glycoside, can also be used. For example the attachment to the O atom of a —CH(OH)— alcohol group can take place with formation of a —CH(OR)— group. The residue R to be attached can for example be a straight-chained or branched alkyl, (—C(O)-alkyl), (—C(O)-alkenyl) or alkenyl residue which preferably comprises at least 6, preferably at least 14, in particular at least 16 carbon atoms, and which in the case of a (—C(O)-alkenyl) or alkenyl residue can have one, two, three or more double bonds. In particular the attachment to the OH group can take place at the C₆-position of the sugar, for example of the glucose component. (The numbering of the carbon atoms in the sugar atoms is known to a person skilled in the art and is for example given in the “Lehrbuch der Organischen Chemie” (supra) by H. Beyer, W. Walter). For example, esterases and/or acylases and/or transesterases and/or transferases, in particular transferases transferring acyl groups, can be used as enzyme, in particular if they display the activity described above for attaching an alkyl, (—C(O)-alkyl), -C(O)-alkenyl) or alkenyl residue. As examples, pig-liver esterase (PLE, EC 3.1.1.3), penicillin acylase (EC 3.5.1.11) or different amino acid acylases (EC 3.5.1.14) may be named here.

A transformation of a glycoside with attachment of an alkyl, (—C(O)-alkyl), (—C(O)-alkenyl) or alkenyl residue, can take place in particular in the case of glycosides, in particular sterylglycosides or glucosides, in which a fatty-acid residue was present at the OH group in the C₆-position of the sugar which, upon transformation of the fats, in particular by transesterification, for example with methanol, was lost accompanied by formation of a more poorly soluble non-acylated sterylglycoside. When using esterases and/or acylases and/or transesterases and/or transferases it is in particular advantageous if the biodiesel, biodiesel precursors or fats already contain a not inconsiderable quantity of fatty acids as impurities. This means that in this case a substrate for the enzyme is already in situ which can attach to the OH group at the C₆-position of the sugar component of the glycoside, in particular of the glucose component of a glucoside.

Particularly advantageously, mixtures of several enzymes with the same or different activity may also be used.

After enzymatic incubation an obtained transformation product of the glycoside or the at least one cleavage product of the glycoside can have a higher solubility in the biodiesel, the biodiesel precursor, the vegetable or animal fats, as well as their mixtures, than the glycoside itself. This is the case in particular if the glycoside included a hydrophobic section or a section with hydrophobic zones which, after enzymatic cleavage, has a higher solubility in the biodiesel, vegetable or animal fats, as well as their mixtures, than the glycoside present before the enzymatic incubation. For example the cleavage products obtained after cleavage from a sterylglycoside which contain the steryl residue or sections of the steryl residue have a higher solubility in biodiesel or precursors.

A transformation product of the glycoside obtained after the enzyme incubation or the at least one cleavage product of the glycoside can have a lower solubility in the biodiesel, vegetable or animal fats or their mixtures than the glycoside itself and then be separated. This is the case in particular if the glycoside comprises a section which is hydrophilic or less lipophilic compared with other sections or a section with hydrophilic or relatively less lipophilic zones which, after enzymatic cleavage, has a lower solubility in the biodiesel or the biodiesel precursors, as well as their mixtures, than the glycoside present before the enzymatic incubation. For example this can be the case if the enzyme cleaves a compound with at least one sugar portion or component, in particular an aldo- or ketohexose component, from a glycoside, in particular a sterylglycoside.

The separation of the transformation product of the glycoside or of the at least one cleavage product of the glycoside can then for example advantageously take place in each case by sedimentation or filtration, in particular, but not exclusively, without prior cooling.

It is also advantageous, according to an embodiment according to aspects of the invention, if a separation of the transformation product of the glycoside and/or of the at least one cleavage product of the glycoside can take place by transfer into a solvent phase in which the solubility of the latter is higher than in the biodiesel or the biodiesel precursor, or the mixture thereof.

Any solvent known to a person skilled in the art on the basis of his knowledge which, after thorough mixing with biodiesel or biodiesel precursor or their mixtures, separates again from these at least after several hours and forms a phase above or below the phase which comprises the largest part of the biodiesel to be purified or the biodiesel precursor or their mixtures can be chosen as solvent phase.

According to a preferred embodiment it can in particular be advantageous if, before use in the method according to aspects of the invention, the solvent phase is an aqueous phase which consists of water or comprises more than 50 wt.-%, preferably more than 85 wt.-%, preferably more than 95 wt.-% water, relative to the total weight of the solvent phase. Aqueous solvent phases before use in the method according to aspects of the invention are also called aqueous purification mixtures below. Further constituents can be any additives selected on the basis of the knowledge of a person skilled in the art, for example one or more organic solvents, salts of organic or inorganic acids or bases, organic and inorganic acids, bases, pH-buffering mixtures, phase-transfer excipients, detergents as well as one or more enzymes.

Preferably, before use in the method according to aspects of the invention, the solvent phase is water or an aqueous purification mixture, as explained below.

In many applications, it has proved advantageous in particular if, before use in the method according to aspects of the invention, the solvent phase is a solution or suspension which comprises water and at least one water-soluble enzyme.

The quantity of the at least one enzyme relative to the quantity of water can for example lie in a range of from 0.01-20 g per litre.

According to a preferred embodiment, the method according to aspects of the invention comprises the following steps:
providing a biodiesel or a biodiesel precursor or mixtures thereof, containing at least one glycoside,
providing at least one enzyme which can cleave or transform the at least one glycoside, in particular in the form of an aqueous enzyme solution;
incubating the biodiesel, the biodiesel precursor or the mixture thereof with the at least one enzyme, in order to cleave or transform at least a part of the at least one glycoside;
separating the at least one enzyme or the enzyme solution, preferably with at least one cleavage or transformation product of the glycoside which has a lower solubility in the biodiesel, the biodiesel precursor or the mixture thereof than the glycoside itself.

Furthermore, according to a preferred feature, no filtration, in particular no ultrafiltration, is carried out.

The present invention also provides an embodiment of the method according to aspects of the invention which is extremely advantageous and which enables the advantages of purification via a two-phase system to be exploited. According to this embodiment of the method according to aspects of the invention the step of incubating with the at least one enzyme comprises several steps, wherein in a first step, a bringing into contact of a starting mixture which comprises biodiesel or biodiesel precursors or a mixture of these, and which also contains one or more glycosides, with water or an aqueous purification mixture takes place. Here, either the starting mixture or preferably the aqueous purification mixture contains at least one enzyme. The bringing into contact also takes place over an adequate period of time in order to at least partly transform or cleave the one or more glycosides by the at least one enzyme. The duration of this period of time can be ascertained by a person skilled in the art by simple tests on the basis of his general knowledge, for example by tracking the sugar content of the aqueous phase, as explained below. The reaction temperature can lie between 15° C. and 80° C., preferably 30° C. and 60° C., the reaction time between 1 min and 24 hours, preferably 20 and 240 min. The thorough mixing can take place by means of stirring or pumping.

After the transformation or cleavage of the glycoside has been achieved to the desired extent, in a subsequent step a separation of a phase, i.e. an aqueous mixture, from the purified starting mixture, takes place. There is now at least one transformation product of the glycoside or at least one cleavage product of the glycoside in the separated aqueous phase. In this way relatively hydrophilic transformation or cleavage products can very easily be removed from the mixture.

According to a preferred feature of this invention the at least one enzyme is also partly or completely soluble in water. This has the advantage that all, or essentially all, of this or these enzyme(s) remain(s) in an aqueous phase once this has been brought into contact with a phase comprising biodiesel or biodiesel precursors or their mixtures. If they essentially all remain in an aqueous phase, this means that more than 90%, preferably more than 96%, preferably more than 99% of the quantity of enzyme remains in the aqueous phase.

It was possible to obtain particularly advantageous results if the process according to aspects of the invention is carried out in a two-phase system comprising biodiesel (and/or biodiesel precursor and/or their mixtures) and aqueous phase in which a β-glucosidase is dissolved.

The use of a two- (multi-) phase system firstly makes possible a simple separation of transformation or cleavage products of the glycoside. Additionally, it is thereby ensured that no, or essentially no, enzyme or parts of the enzyme preparation enters the mixture to be purified and can then potentially lead to problems in the end product, the fuel based on renewable raw materials. The use of a two-phase system, starting from a continuous oil phase or lipophilic phase with the biodiesel, the biodiesel precursor or the mixture thereof (and not a suspension, or the like, in particular not a lecithin-containing suspension) and a continuous aqueous phase with the at least one enzyme, is particularly preferred.

After concluding the separation step, a purified mixture which contains biodiesel, biodiesel precursor,, vegetable or animal fats or their mixtures and contains less of one or more glycosides than the starting mixture is produced with the method according to aspects of the invention.

It is assumed, without the present invention being bound by the correctness of this assumption, that the glycosides which comprise hydrophilic and hydrophobic portions, for example sterylglycosides, form at the biodiesel/water interface (and/or biodiesel precursor/water interface), which makes it possible for the enzyme, for example a β-glucosidase, dissolved in the aqueous phase to cleave off the sugar residue, which then dissolves in the water. The hydrophobic glycosyl residue, for example the steryl residue, remains dissolved in the biodiesel.

The above assumptions are confirmed by the fact that a formation of sugars, such as for example glucose starting from sitosteryl-β-glucoside, can be demonstrated by a standard test for determining reducing sugars, such as for example the DNSS method or with Fehling's solution. Further methods for determining sugars are known to a person skilled in the art and can be selected according to their specific application.

Advantageous results can for example be obtained upon separation of glucosides, and in particular of sterylglucosides.

The method can also, before or after the step of incubating, comprise a further washing step, for example a so-called water wash, or the incubation can be integrated into the water wash customarily taking place during the production of biodiesel.

According to one possible embodiment, biodiesel is subjected to a water wash during its production. The water wash can take place in one or several stages. In the water wash, water is added to the crude biodiesel, wherein the quantity of water relative to the biodiesel is chosen in the range of from preferably 2 to 10 wt.-%, preferably 4 to 8 wt.-%. During the water wash the mixture is lightly moved, wherein the intensity of the motion is chosen to be such that no stable emulsion forms. The temperature of the water phase is preferably chosen in the range of from 20 to 90° C., particularly preferably 40 to 80° C. The duration of the treatment of the biodiesel with water depends on the quantities chosen. Preferably the duration is chosen in the range of from 5 to 45 minutes. The water wash step is preferably repeated at least once after separation of the aqueous phase, wherein the water quantity and the water temperature can also be chosen to be different from the first water wash step. After separating the aqueous phase or after concluding the water wash, the oil is preferably dried. For this, the biodiesel can for example be heated, preferably to a temperature of more than 90° C.

Soya oil, palm oil, corn oil, sunflower oil, waste edible fats and oils and/or beef tallow for example can be purified as biodiesel precursor with particularly good results with the method according to aspects of the invention. Likewise for example biodiesel produced partially or completely from soya oil, palm oil, corn oil, sunflower oil and/or beef tallow has proved especially advantageous for purifying. Also, the method according to aspects of the invention can be used with very good results for purifying fats which are contaminated or form as waste products, for example frying oils.

The crude biodiesel used in the method according to aspects of the invention is preferably obtained by alcoholysis of triglycerides. The triglycerides can be obtained as such from any suitable source of oils and fats. The alcoholysis is carried out according to methods known per se, wherein acid or, preferably, alkaline catalysts can be used. Methanol is preferably used as alcohol. However, it is also possible to use other alcohols, for example ethanol or propanol. Ethanol offers the possibility of obtaining the biodiesel completely from biological sources, because ethanol can be obtained by fermenting an organic substance.

Soya oil and palm oil in particular, as well as biodiesel or biodiesel precursors made of soya oil and palm oil, are affected by the danger of a formation of sterylglycoside precipitates, because of the high concentration of sterylglycosides present therein. Specifically with these biodiesels, problematic from the point of view of a formation of precipitates, particularly good results were able to be achieved with the method according to aspects of the invention, and the danger of a potential formation of precipitates countered. A removal of sterylglycosides is therefore also advantageous in particular because these can encourage or induce a precipitation of other substances.

The glycoside to be at least partially cleaved or transformed by the method according to aspects of the invention can in particular be a glucoside, mannoside or fructoside. Particularly good results have been obtained with the method according to aspects of the invention when separating sterylglycosides, preferably sterylglucosides, preferably sitosteryl, stigmasterol and campesterol betaglucosides.

The present invention also teaches in particular, as. described in the introduction, the use of at least one enzyme which can cleave or transform glycoside, for purifying biodiesel, biodiesel precursors, vegetable or animal fats, as well as their mixtures.

Biodiesel or biodiesel precursors as well as their mixtures which can be obtained according to the method according to aspects of the invention contain fewer glycosides, in particular sterylglycosides, and are thus less inclined to form blockages or deposits. The present invention therefore provides the consumer with better-quality fuels based on renewable raw materials.

According to a particularly preferred further feature the invention therefore relates to the use of at least one enzyme which can cleave or transform glycoside, for improving the storage stability and/or the filterability of biodiesel, or of a biodiesel precursor as well as their mixtures, in particular at temperatures below 40° C., preferably below 30° C., quite particularly preferably below 20° C. Thus in the so-called “filter blocking” tests it was shown that the treatment according to aspects of the invention of biodiesel, a biodiesel precursor as well as their mixtures with at least one enzyme which can cleave or transform glycoside can delay or prevent a blocking of the filter. Suitable filter blocking tests are described for example in WO 2007/076163 A2, the disclosure of which in this regard is included by reference in the present description. Thus according to possible embodiments such tests include the test described at the bottom of page 5/top of page 6 of WO 2007/076163 A2, the methods according to IP387 and ASTM D2068 and a modified ASTM 6217 test according to pages 13 to 15 of WO 2007/076163 A2.

Methods

The following methods are used to determine the parameters of the method according to aspects of the invention:

1. Enzymes

β-glucosidases from various organisms, naringinase, cellulase, β-glucanase, lactase and various hemicellulases, all available from ASA Spezialenzyme GmbH, Wolfenbüttel, Del., were used as enzymes.

2. Measurement Methods

The enzymatic cleavage of the sterylglycosides was measured according to the following method: the enzyme tested in each case was dissolved or dispersed in an aqueous buffer mixture. One portion by volume of this aqueous buffer/enzyme mixture was mixed with 10 portions by volume biodiesel in a beaker or test tube (depending on the size of the volume) and incubated accompanied by stirring on the magnetic stirrer with the corresponding reaction times and temperatures. After the reaction was ended the aqueous phase was separated off with a separating funnel or by centrifuging and the sterylglycoside content of the biodiesel examined by means of HPLC. The water content and the acid number were also determined. The reducing sugars were measured by means of the DNSS method in the aqueous subphase. As controls,

a) the biodiesel was treated with buffer only but without enzyme

b) the enzyme/buffer mixture was treated accordingly with water instead of biodiesel

with the corresponding reaction times and temperatures, the quantity of the reducing sugars determined in the subphase and these values deducted as blind values from the values achieved with the enzyme/buffer/biodiesel mixtures.

3. DNSS Method: Determining the Glucose Content (Measuring the Reducing Sugars)

The test method follows instructions from ASA Spezialenzyme GmbH, D-38302 Wolfenbüttel, Germany.

Test principle: Glucose is photometrically detected with 3,5-dinitrosalicylic acid (DNSS). Upon heating of 3,5-dinitrosalicylic acid (DNSS) with reducing sugars in an alkaline medium, 3-amino-5-nitrosalicylic acid forms. A nitro group is reduced to the amino group, while the aldehyde group of the monosaccharide is oxidized to the carboxyl group (Kakac, B., Vejdelek, Z. J.: Handbuch der photometrischen Analyse, Vol. I, Verlag Chemie, Weinheim 1974). Different sugars produce different colours, the absorption maximum of which lies between 500 and 550 nm. Thus mono-, di-, oligo- and polysaccharides as well as methylpentoses and O-methylsaccharides can also be covered by this test.

Reagents:

DNSS phenol reagent: Solution A: 38.55 g K—Na tartrate is weighed into a 200 mL beaker and dissolved in 125 mL dist. water, 2.425 g NaOH (platelets) is dissolved in the solution.

Solution B: 1.325 g 3,5-dinitrosalicylic acid (C7H₄N₂O₇; 2-hydroxy-3,5-dinitro benzoic acid) is dissolved in 125 mL dist. water in a brown screw-cap bottle.

Solution C: 1.05 g phenol is dissolved in 12.5 mL dist. water. 0.25 g NaOH (platelets) and 1.05 g Na₂SO₄ are added successively and dissolved accompanied by stirring.

Working solution: Solution A and solution C are poured (without subsequent rinsing) into solution B and homogenized for 10 min. The solution is left to stand for at least one night prior to use and kept in the dark at all times.

Standard solution: 2.0 g/L grape sugar (glucose) in dist. water.

Procedure: Samples: 1.0 mL sample solution is mixed with 2.0 mL DNSS/phenol reagent and boiled for 5 min. The mixture is cooled for approx. 5 min in the ice bath and the absorbance measured at 546 nm and room temperature.

Blind values: 1.0 mL water is mixed with 2.0 mL DNSS/phenol reagent and boiled for 5 min. The mixture is cooled for approx. 5 min in the ice bath and the absorbance measured at 546 nm and room temperature.

Standard: 1.0 mL dil. standard solution (see calibration table) is mixed with 2.0 mL DNSS/phenol reagent and boiled for 5 min. The mixture is cooled for approx. 5 min in the ice bath and the absorbance measured at 546 nm and room temperature.

Calibration: Dilutions of the standard solution to produce the calibration lines:

| c (glucose) | standard | dist. water |
|---|---|---|
| in g/l | sln. in μl | in μl |
| 0.1 | 50 | 950 |
| 0.12 | 60 | 940 |
| 0.16 | 80 | 920 |
| 0.24 | 120 | 880 |
| 0.28 | 140 | 860 |
| 0.32 | 160 | 840 |

4. Measuring the Sterylglycosides with HPLC:

A GC-MS method developed by ASG Analytik-Service Gesellschaft mbH, Trentiner Ring 30, 86356 NeusäB was used for measuring the sterylglycoside. The procedure is as follows:

1. Concentration of the sterylglucoside with the IP 387/97 filter blocking tendency (FBT test) on a 1.6 μm fibreglass filter. Measurement of the volume of biodiesel flowing through the filter. (Approx. 300 mL biodiesel is required for a complete test.)

The filter is extracted from the FBT test with 4 mL hexane.

The sterylglucosides insoluble in hexane are dissolved with 1 mL pyridine.

100 μL MSTFA as silylation reagent and 50 μL tricaprine solution (71.3 mg tricaprine on 10 mL pyridine) are added.

The mixture is left to stand for 20 min at 60° C.

7 mL hexane is added.

Solution is filtered over a 0.45 μm injection filter and 1 μL of this injected into the GC/MS system.

2. Calibration Standards

The quantification takes place with external calibration 1. A parent solution is produced from a pure sterylglucoside mixture in pyridine (approx. 50 mg/10 mL).

2. Various volumes (between 10 and 100 μL) of the parent solution are pipetted off.

3. 100 μL MSTFA as silylation reagent and 50 μL tricaprine solution (71.3 mg tricaprine on 10 mL pyridine) are added.

4. The mixture is left to stand for 20 min at 60° C.

5. 8 mL hexane is added.

6. Solution is filtered over a 0.45 μm injection filter and 1 μL of this injected into the GC/MS system.

3. GC/MS Measurement

3.1 GC Conditions

Precolumn: Zebron Guard Column; 10 m; 0.32 mm ID

Column: Zebron-5HT Inferno; 15 m; 0.32 mm ID; 0.25 μm

| | |
|---|---|
| Injection: | on column |
| Carrier gas: | helium |
| Flow: | 1.5 ml/min |

Oven: Temperature kept at 60° C. for 1 min, heated at 15° C./min to 375° C., held for 3 min

3.2 MS conditions

| Segment 1: | 0-2 min hexane, cut-off | |
|---|---|---|
| Segment 2: | 2-25 min EI (auto), 40-650 m/z | |
| Scan time: | 0.50 | scans/sec |
| Multiplier Offset: | 0 | V |
| Emission current: | 40 | μA |
| Count threshold: | 1 | count |
| Target TIC: | 10000 | counts |
| Prescan ionization time: | 100 | μsec |
| Max. ionization time: | 5000 | μsec |
| Background mass: | 50 | m/z |
| RF dump value: | 650 | m/z |

4. Evaluation

The sterylglucosides are identified with the help of GC/MS and quantified with the external calibration in the samples.

5. Biodiesel Used

A biodiesel (methyl ester) which had been produced from palm oil was used for the tests described below. A sterylglycoside content of 50 ppm was able to be recorded by means of HPLC in the starting sample. At room temperature the sterylglycosides are visible in the form of cloudings of small crystals and flakes. These precipitations disappear when the sample is heated to 80° C. After cooling, these drop out again, i.e. the process is reversible. Experience shows that this applies only if the water content of the biodiesel is low.

The invention is explained in more detail below with reference to the following, non-limiting examples:

### EXAMPLES

### Example 1

The enzymes β-glucosidase 1 (enzyme 1) and 2 (enzyme 2) were dissolved in 80 ml 0.05 M Na-citrate buffer, pH 5.0, and mixed with 800 ml sterylglycoside biodiesel.

This mixture was poured into a 1 L beaker and incubated on the magnetic stirrer for 1 hr at 37° C. accompanied by stirring.

After the reaction was ended, the aqueous phase was separated off with a separating funnel and the sterylglycoside content of the biodiesel examined by means of HPLC. The water content and the acid number were also determined. The reducing sugars were measured in the aqueous subphase by means of the DNSS method.

The results are listed in the following tables:

TABLE 1

| Sample name | SC [ppm] |
|---|---|
| Biodiesel, untreated | 10 |
| BD with enzyme 2, 1.2 g/800 ml | <DL |
| BD with enzyme 1, 1.2 g/800 ml | <DL |
| BD with enzyme 1, 0.6 g/800 ml | <DL |
| BD with enzyme 1, 0.3 g/800 ml | <DL |

Table 1 shows a comparison overview of the sterylglycoside content (“SC”, given in [ppm]; “<DL”: below the detection limit).

The data show that the sterylglycoside content of the biodiesel sample is reduced by the different enzymatic treatments from 10 ppm to below the detection limit.

### Example 2

The enzyme β-glucosidase 1 is dissolved in different concentrations in 0.3 ml aqueous 0.05 M citrate buffer, pH 5.0, and treated with 3 ml biodiesel as in Methods, 2. Measurement methods.

The results in Table 2 show that increasing quantities of reducing sugars are extracted into the aqueous subphase as the enzyme dose increases.

TABLE 2
Enzymatic cleavage of sterolglycosides

| | | c_{enzyme (aqueous} | T | Inc. time | Red. sugar |
|---|---|---|---|---|---|
| No. | Enzyme | ₚₕₐₛₑ₎ [g/L] | [° C.] | [min] | in SP [g/L] |
| | β-glucosidase 1 | 0.8 | 37 | 60 | 0.1 |
| | β-glucosidase 1 | 1.6 | | | 0.2 |
| | β-glucosidase 1 | 8.0 | | | 2.0 |
| | β-glucosidase 1 | 16.0 | | | 3.2 |

C_{enzyme (aqueous phase)}: concentration of the enzyme relative to the volume of the aqueous phase

### Example 3

The enzyme β-glucosidase 1 is dissolved with a concentration of 1.6 g/L in 0.3 ml aqueous 0.05 M citrate buffer, pH 5.0, and incubated with 3 ml biodiesel at different reaction times, otherwise treated as described under Methods, 2. Measurement methods.

The results in Table 3 show that increasing quantities of reducing sugars are extracted in the aqueous subphase as the reaction time increases.

TABLE 3
Enzymatic cleavage of sterolglycosides

| | | c_{enzyme (aqueous} | T | Inc. time | Red. sugar |
|---|---|---|---|---|---|
| No. | Enzyme | ₚₕₐₛₑ₎ [g/L] | [° C.] | [min] | in SP [g/L] |
| | β-glucosidase 1 | 1.6 | 37 | 0 | 0 |
| | | | | 30 | 0.2 |
| | | | | 60 | 0.4 |
| | | | | 105 | 0.4 |
| | | | | 150 | 0.5 |

C_{enzyme (aqueous phase)}: concentration of the enzyme relative to the volume of the aqueous phase

### Example 4

Different enzymes are dissolved in different concentrations in 0.3 ml aqueous 0.05 M citrate buffer, pH 5.0, and treated with 3 ml biodiesel as in Methods, 2. Measurement methods.

Table 4 shows that, in addition to beta-glucosidase, numerous other enzymes can be used in the method according to aspects of the invention. In particular beta-glucanase, naringinase and cellulose are suitable.

TABLE 4
Enzymatic cleavage of sterolglycosides

| | | c_{enzyme (aqueous} | T | Inc. time | Red. sugar |
|---|---|---|---|---|---|
| No. | Enzyme | ₚₕₐₛₑ₎ [g/L] | [° C.] | [min] | in SP [g/L] |
| | β-glucosidase 1 | 8.0 | 37 | 60 | 2.3 |
| | β-glucosidase 3 | 16.0 | | | 0.5 |
| | β-glucanase | 16.0 | | | 0.8 |
| | Naringinase | 16.0 | | | 2.2 |
| | β-glucosidase 2 | 16.0 | | | 1.9 |
| | Cellulase | 16.0 | | | 0.3 |
| | powder 1 | | | | |
| | Cellulase | 16.0 | | | 0.4 |
| | powder 2 | | | | |

c_{enzyme (aqueous phase)}: concentration of the enzyme relative to the volume of the aqueous phase

### Example 5

The method according to aspects of the invention can be used not only for cleaving sterylglycosides from biodiesel but also for cleaving sterylglycosides from vegetable oils. The procedure is analogous in this case.

1. Method for purifying biodiesel, a biodiesel precursor or their mixtures, containing at least one glycoside, wherein the biodiesel or the biodiesel precursor or the mixture thereof has a lecithin content of less than 10 wt.-% and is incubated with at least one enzyme in order to transform or to cleave the at least one glycoside.

2. Method according to claim 1, wherein the transformation product of the glycoside or at least one cleavage product of the glycoside obtained after the enzyme incubation has a higher solubility in the biodiesel or the biodiesel precursor or the mixture thereof than the glycoside itself.

3. Method according to claim 1, wherein the transformation product of the glycoside or at least one cleavage product of the glycoside obtained after the enzyme incubation has a lower solubility in the biodiesel or the biodiesel precursor or the mixture thereof than the glycoside itself and is then separated off.

4. Method according to claim 3, wherein the separation takes place via sedimentation, filtration or transfer into a solvent phase in which the transformation product of the glycoside or at least one cleavage product of the glycoside has a higher solubility than in the biodiesel or the biodiesel precursor or the mixture thereof.

5. Method according to claim 1, wherein the biodiesel precursor represents oil or fat, in particular degummed and/or deodorized oil or fat, or one such oil or fat was used for producing the biodiesel, wherein preferably the lecithin content of the fat or oil or biodiesel is less than 5 wt.-%, more preferably less than 10 ppm, in particular less than 5 ppm.

6. Method according to claim 1, comprising the following steps:
a. providing a biodiesel or a biodiesel precursor or mixtures thereof, containing at least one glycoside,
b. providing at least one enzyme which can cleave or transform the at least one glycoside, in particular in the form of an aqueous enzyme solution;
c. incubating the biodiesel, the biodiesel precursor or the mixture thereof with the at least one enzyme, in order to cleave or transform at least a part of the at least one glycoside;
d. separating the at least one enzyme or the enzyme solution, preferably with at least one cleavage or transformation product of the glycoside which has a lower solubility in the biodiesel, the biodiesel precursor or the mixture thereof than the glycoside itself.

7. Method according to claim 1, wherein no filtration, in particular no ultrafiltration, is carried out.

8. Method according to claim 1, wherein the step of incubating with the at least one enzyme comprises:
bringing a starting mixture which comprises biodiesel or a biodiesel precursor or a mixture thereof, as well as containing one or more glycoside, into contact with water or an aqueous purification mixture,
wherein either the starting mixture or the aqueous purification mixture comprises at least one enzyme, and wherein the bringing into contact takes place over a sufficient period of time in order to at least partly transform or cleave the one or more glycoside by the at least one enzyme;
separating an aqueous mixture from the purified starting mixture, wherein the aqueous mixture comprises water and at least one transformation product of the glycoside or at least one cleavage product of the glycoside.

9. Method according to claim 1, wherein the at least one enzyme is partly and/or completely soluble in water and/or completely or essentially completely remains in an aqueous phase, after this was brought into contact with a phase, comprising biodiesel or a biodiesel precursor or their mixtures.

10. Method according to claim 1, wherein the method comprises a further wash step before and/or after the step of incubation.

11. Method according to claim 1, wherein the at least one enzyme is an acylase, esterase, transesterase, transferase or hydrolase, in particular glycosidase, wherein the enzyme is preferably selected from the group consisting of glucosidases, in particular beta-glucosidases, naringinases, β-glucanases, chitinases and cellulases.

12. Method according to claim 1, wherein the biodiesel precursor is selected from the group consisting of soya oil, palm oil, corn oil, sunflower oil, beef tallow, frying fat, other waste edible fats and oils and their mixtures and/or wherein the biodiesel is produced partly or completely from one of the vegetable or animal fats, selected from the group consisting of soya oil, palm oil, corn oil, sunflower oil, beef tallow, frying fat, other waste edible fats and oils and their mixtures
and/or wherein the glycoside is selected from the group consisting of glucosides, mannosides, fructosides and their mixtures and/or wherein the glycoside is selected from the group consisting of sterylglycosides, preferably sterylglucosides, preferably sitosteryl, stigmasterol and campesterol betaglucosides.

13. A method comprising the use of at least one enzyme which can cleave or transform glycoside for purifying biodiesel or a biodiesel precursor as well as their mixtures.

14. Method according to claim 13 for improving the storage stability and/or the filterability of biodiesel, or a biodiesel precursor as well as their mixtures, in particular at temperatures below 40° C., particularly preferably below 30° C., quite particularly preferably below 20° C.

15. Biodiesel or biodiesel precursor or their mixtures which can be obtained according to the method according to claim 1.
